(19) United States
(12) Patent Application Publication
(10) Pub. No.: US 2021/0401607 A1
(43) Pub. Date: Dec. 30, 2021

(54) Elbow and Forearm Orthosis

(71) Applicant: Jeffrey Peter Sanderson, Victoria (AU)

(72) Inventors: Jeffrey Peter Sanderson, Victoria (AU); Brodwen McBrain, Victoria (AU)

(21) Appl. No.: 17/285,696

(22) PCT Filed: Oct. 15, 2019
(86) PCT No.: PCT/AU2019/000128

§ 371 Date: Apr. 15, 2021

(30) Foreign Application Priority Data
Oct. 15, 2018 (AU) 2018903897

(51) Int. Cl. A61F 5/01

(52) U.S. Cl. CPC A61F 5/013; A61F 2005/0158; A61F 2005/0165

(57) ABSTRACT

There is described an orthosis comprising: a first portion adapted to be secured to an upper arm region of a wearer; a second portion hingedly connected to the first portion and comprising a pair of elongate members spaced apart to extend along opposing sides of a lower arm region of the wearer, each of the elongate members terminate at a distal end adjacent to a wrist of the wearer and being connected by a connecter extending at least partially around the wrist of the wearer; wherein, the distal ends of the elongated members each have a connector element formed therein for facilitating engagement with the wearer's hand so as to maintain the wearer's hand in either a pronated or a supinated position.

### RELATED APPLICATIONS

The present application claims priority from the Applicant's earlier filed Australian provisional patent application no. 2018903897 filed 15 Oct. 2018, the entire ontents of which are incorporated herein by reference.

### FIELD OF INVENTION

This invention relates generally to an orthotic device for the elbow, forearm and wrist, and in particular to an orthotic device that can be simply and effectively employed to apply a variety of corrective forces to a wearer's lower arm region to compensate or stretch for lost or limited movement.

### BACKGROUND OF THE INVENTION

Forearm rotation, as well as elbow flexion and extension movements, are critical functions for the placement of the hand in space. Limitations in movement of any one or more of these directions can have a significant impact on a person's ability to complete the simple activities of daily living, as well as their ability to participate in the community and workforce.

Injury to the elbow, forearm or wrist commonly results in loss or stiffness of one or a number of these movements. Evidence has shown that in such situations, the provision of a sustained stretch for the affected joint can improve the range of motion over time.

In other cases, injury or surgical rehabilitation protocols can result in the need to limit or assist one or a number of these movements in order to reduce stress on healing structures. Additionally, injury or disability may limit movement/s in certain directions, thereby requiring a means to facilitate movement in those specific directions.

A number of orthoses have been developed for use in either stretching or protecting structures of the elbow, forearm and/or wrist, with varying degrees of success. Some proposed orthoses act on rotation (supination and/or pronation) and thus immobilise the elbow, which is not desirable as it results in otherwise unnecessary stiffness and loss of function of the wearer's arm. Many other proposed orthoses cover the wearer's hand or have a protruding profile that further impedes function during wear. Also, the forces applied by many of the existing orthoses are of limited strength to sufficiently stretch or limit movement in the direction(s) desired. No existing orthosis is able to control or move through both elbow flexion and extension and supination and pronation arcs simultaneously.

Thus, there is a need to provide for an improved orthosis that can be adaptable for a variety of purposes whereby it can be used to facilitate movement/s in desired direction/s and to control any unwanted forces and movements on healing structures while further leaving the hand free to function.

The above references to and descriptions of prior proposals or products are not intended to be, and are not to be construed as, statements or admissions of common general knowledge in the art. In particular, the above prior art discussion does not relate to what is commonly or well known by the person skilled in the art, but assists in the understanding of the inventive step of the present invention of which the identification of pertinent prior art proposals is but one part.

### SUMMARY OF THE INVENTION

Accordingly, in one aspect of the invention there is provided an orthosis comprising:
a first portion adapted to be secured to an upper arm region of a wearer;
a second portion hingedly connected to the first portion and comprising a pair of elongate members spaced apart to extend along opposing sides of a forearm region of the wearer, each of the elongate members terminate at a distal end adjacent to a wrist region of the wearer and being connected by a connecter extending at least partially around the wrist region of the wearer;
wherein, the distal ends of the elongated members each have a connector element formed therein for facilitating engagement with the wearer's hand so as to maintain the wearer's hand in either a pronated or a supinated position.

In one embodiment, the wearer's hand comprises a wrist worn element having one or more attachment members extending from opposing sides thereof for engaging with the connector elements of the elongate members so as to maintain the wearer's wrist in either a pronated or a supinated position when relaxed.

The wrist worn element may be a soft orthosis. In another embodiment, the wrist worn element may be a semi rigid orthosis. In yet another embodiment, the wrist worn element may be a rigid orthosis. In yet another embodiment, the wrist worn element may be a forearm worn cuff.

The one or more attachment members may be elastic. The elastic attachment members may allow movement of the wearer's wrist in an opposing direction when the wearer is active and return to the desired supination or pronation position when the wearer is relaxed.

The one or more attachment members may be substantially inelastic or are taut elastic. The inelastic attachment members may maintain the wearer's wrist in a chosen degree/s of rotation including supinated only, pronated only, restricted supination, restricted pronation or restricted in both supination and pronation.

The wrist worn element may be a wrist cuff having one or more elastic attachment members extending from opposing sides thereof for engaging with the connector elements of the elongated members so as to maintain the wearer's wrist in either a pronated or a supinated position when relaxed. The elastic attachment members may allow movement of the wrist in an opposing direction when the wearer is active and return the wrist to a desired supination or pronation when relaxed.

The wrist worn element may be a soft wrist cuff having one or more inelastic or taut elastic attachment members extending from opposing sides thereof for engaging with the connector elements of the elongated members so as to maintain the wearer's wrist in a chosen degree/s of rotation. The chosen degree of rotation may include supinated only, pronated only, restricted supination, restricted pronation or restricted in both supination and pronation.

The hinged connection between the first and second portions may allow free movement of the elongated members in an elbow flexion and extension plane, thereby allowing free movement of wrist cuff or orthosis in this plane.

The hinged connection between the first and second portions may comprise a flexion spring mechanism for effecting flexion force on the wearer's elbow to maintain the wearer's elbow in flexion and yet allow the wearer to extend their elbow against the flexion force.

The hinged connection between the first and second portions may comprise an extension spring or elastic mechanism for effecting an extension force on the wearer's elbow to maintain the wearer's elbow in extension and yet allow the wearer to flex against the extension force.

The hinged connection between the first and second portions may comprise one or two stopping mechanism/s to limit the wearer's elbow flexion and/or extension so as to limit the wearer's elbow flexion and/or extension and yet allowing elbow movement within the one or two limits.

The hinged connection may be lockable in any desired position of elbow flexion or extension, so as to lock the wearer's elbow in a desired position.

### BRIEF DESCRIPTION OF THE DRAWINGS

The invention may be better understood from the following non-limiting description of preferred embodiments, in which:

FIG. 1 is perspective view of the proximal component of the multipurpose elbow and forearm orthosis in accordance with an embodiment of the present invention;

FIG. 2 is a lateral view of the orthosis of FIG. 1 in use with a wrist orthosis in accordance with an embodiment of the present invention;

FIG. 3 is a plan view of the wrist orthosis of FIG. 2;

FIG. 4 is a perspective view of a wrist cuff for use with the multipurpose elbow and forearm orthosis in accordance with another invention;

FIG. 5 is a lateral view of the multipurpose elbow and forearm orthosis of the present invention configured to apply dynamic elbow flexion and dynamic forearm supination to the wearer;

FIG. 6A and 6B are front views showing the orthosis in mid elbow flexion and depicting how the straps of the orthosis can be employed to effect dynamic supination of the hand;

FIG. 7 is a side view of the multipurpose elbow and forearm orthosis of the present invention being used to effect elbow extension limiting and dynamic forearm supination;

FIG. 8A, 8B and 8C are front views of the multipurpose elbow and forearm orthosis in mid elbow flexion showing: pronation blocked at 0°; supination blocked at 0°; and pronation and supination locked at midrange rotation;

FIG. 9 is a side view of the multipurpose elbow and forearm orthosis of the present invention worn to effect dynamic elbow flexion and dynamic forearm supination;

FIG. 10 is a side view of the multipurpose elbow and forearm orthosis of the present invention worn to effect dynamic elbow flexion and dynamic forearm pronation; and

FIG. 11 is a side view of the multipurpose elbow and forearm orthosis of the present invention worn to effect dynamic elbow extension and dynamic forearm supination.

FIG. 12 is a side view of the multipurpose elbow and forearm orthosis of the present invention worn to effect dynamic elbow flexion with elbow extension block and dynamic forearm supination.

### DETAILED DESCRIPTION OF AN EMBODIMENT OF THE INVENTION

The present invention will be described below in relation to its application for use in controlling and applying movement to a right arm/hand of an individual. It will be appreciated that the orthosis could be applied to any arm of a user requiring treatment irrespective of gender or age.

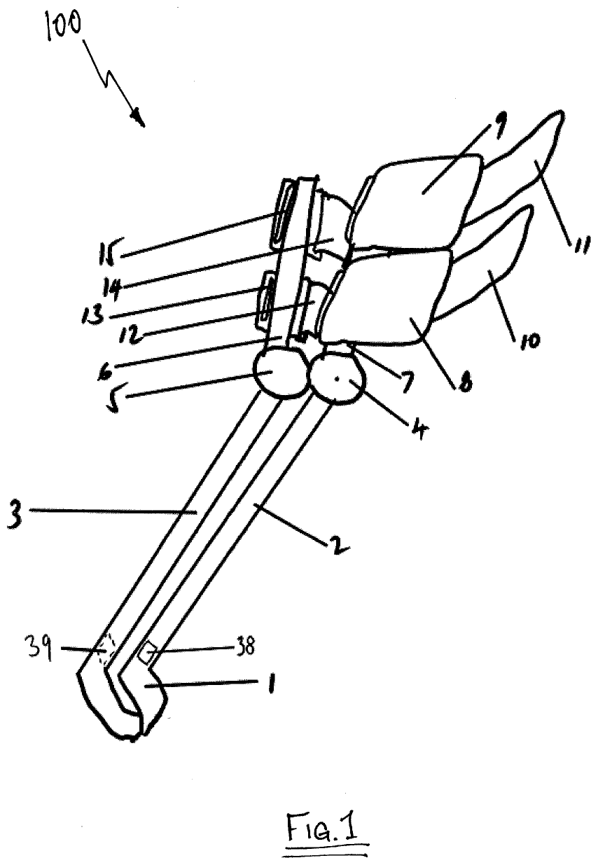

Referring to FIG. 1, the orthosis 100 of the present invention is depicted. The orthosis is configured to be worn about the upper and lower arm of an individual and comprises an upper portion and a lower portion. The upper portion generally comprises a pair of elongate support members 6, 7 that are configured to extend either side of the wearer's humeral bone. The elongate support members (or humeral outriggers) 6, 7 are each attached at their lower ends to a hinge articulation 4, 5, in the form of a disc that enables movement about a central axis thereof. The upper end of the humeral outriggers 6, 7 are free and are attached together by a pair of straps 12, 14 that extend therebetween.

To facilitate attachment of the upper portion to the upper arm, connection straps 10, 11 are provided attached to one of the humeral outriggers 7 at an upper edge thereof. The straps 10, 11 have padding members 8, 9 provided thereon to contact the wearer's arm to minimise rubbing and maximise comfort for the wearer. The upper edge of the other humeral outrigger 6 have loops 13, 15 formed thereon for receiving the straps 10, 11 for enabling the straps 10, 11 to be fastened about the upper arm of the wearer. In this regard, one surface of the straps 10, 11 may be provided with one part of a hook and loop fastener, such as Velcro®, which engages with the other part provided on the other surface of the strap after it has passed through the loop 13, 15. It will be appreciated that when fastened to the upper arm of the user in the manner as described above, the upper portion of the orthosis will be securely yet comfortably located on the wearer's arm for use. Whilst one form of fastening the upper portion of the orthosis to a wearer's arm has been shown, namely by use of a hook and loop fastener material, it will be appreciated that other means for attachment are also envisaged.

The lower portion of the orthosis 100 is positioned to extend about the user's lower arm by way of the pair of elongate support members 2, 3. The elongate support members (or forearm outriggers) 2, 3 are securely attached to opposing hinge articulation members 4, 5 and extend along either sides of the wearer's arm to terminate at a distal end at a point adjacent the wearers hand, wrist or distal forearm. A connector 1 rigidly connects the distal ends of the forearm outriggers 2, 3 to keep the forearm outriggers in alignment with the forearm of the wearer and the connector 1 has a curved configuration to accommodate the wearer's hand. A pair of attachment members 38, 39 are provided on each forearm outrigger 2, 3 immediately adjacent the connector 1 to facilitate secure positioning of the user's hand in a manner that will be described in more detail below.

In a preferred embodiment, the forearm outriggers 2, 3 and the humeral outriggers 6, 7 are formed from a rig material, such as a metal. However, it will also be appreciated that the forearm outriggers 2, 3 and the humeral outriggers 6, 7 may be formed from any suitable rigid material such as a metal or plastic or combination of a metal or plastic material. The manner in which the forearm outriggers 2, 3 and the humeral outriggers 6, 7 attach to the hinge articulation member 4, 5 is such that when attached the angular location of the humeral outrigger compared to the forearm outrigger can vary about the central axis of the hinge articulation member that extends substantially orthogonal to the longitudinal axis of the forearm outriggers 2, 3 and the humeral outriggers 6, 7.

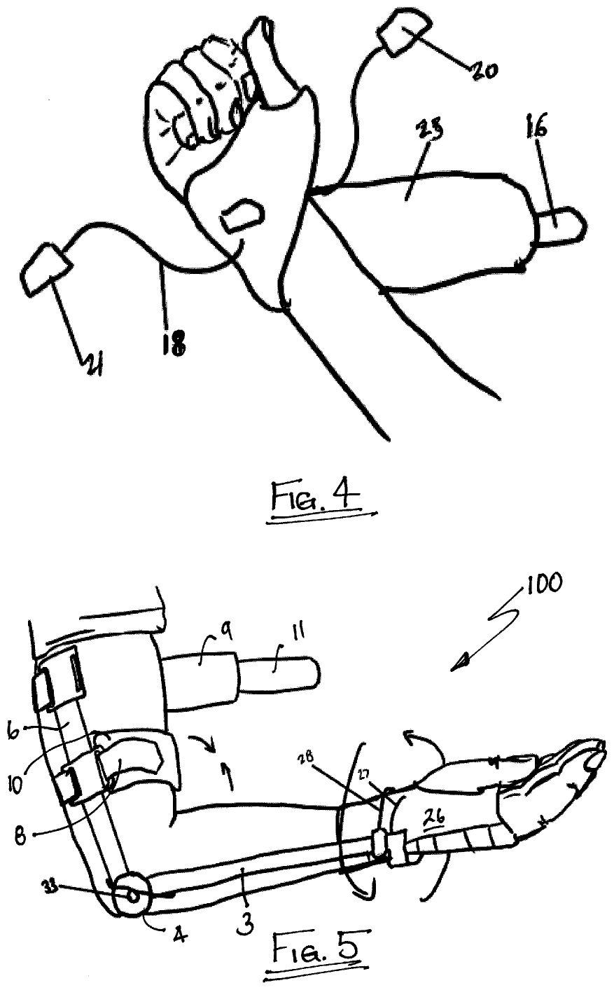

To fit the orthosis 100 to a wearer, the wearer must also be fitted with a wrist worn member, such as a wrist orthosis 26 or wrist cuff 23, as is shown in FIG. 3 and FIG. 4.

Referring to FIG. 3, the wrist orthosis 26 comprises a tubular piece of material having a first hole to accommodate the fingers and a second hole to accommodate the thumb of the wearer such that the wrist orthosis 26 extends over the wearer's wrist and up the hand of the wearer, as shown. A pair of cords 27, 28 extend from the wrist bandage at opposing sides of the wrist which each cord terminating at an attachment element 29, 30. The cords 27, 28 are preferably made from an elastic material. The attachment elements 29, 30 may be hook and loop fastener elements or may be configured to engage with a camlock arrangement formed on the outriggers.

Similarly, as is shown in FIG. 4, the wrist cuff 23 may be employed which is in the form of a wrap member having a thumb hole for receiving the wearer's thumb. The remainder of the body of the cuff 23 is then wrapped about the wearer's wrist and secured in position about the wearer's wrist by way of fastener member 16, which may be a hook and loop fastener. A pair of cords 18, 19 extend from the wrist cuff 23 at opposing sides of the wrist which each cord 18, 19 terminating at an attachment element 20, 21. The cords 18, 19 are preferably made from an elastic material. In the case where the cords are to be used to restrict movement or lock the wearer in a supination or pronation position, the cords may be made from an inelastic material. In such embodiment to be described below, the reference numerals 34, 35 may be used to refer to such inelastic cords.

Whilst a wrist bandage 26 and wrist cuff 23 have been described above as components to be worn by the wearer for fitting the orthosis 100, other hand or wrist or forearm worn bands may also be used for this purpose. The forearm outriggers 2, 3 have attachments 38, 39 which facilitate connection of the cords of the wrist component, such as the wrist orthosis 26 or wrist cuff 23. This allows dynamic, static or other forces to be applied to the wearer in the rotational plane.

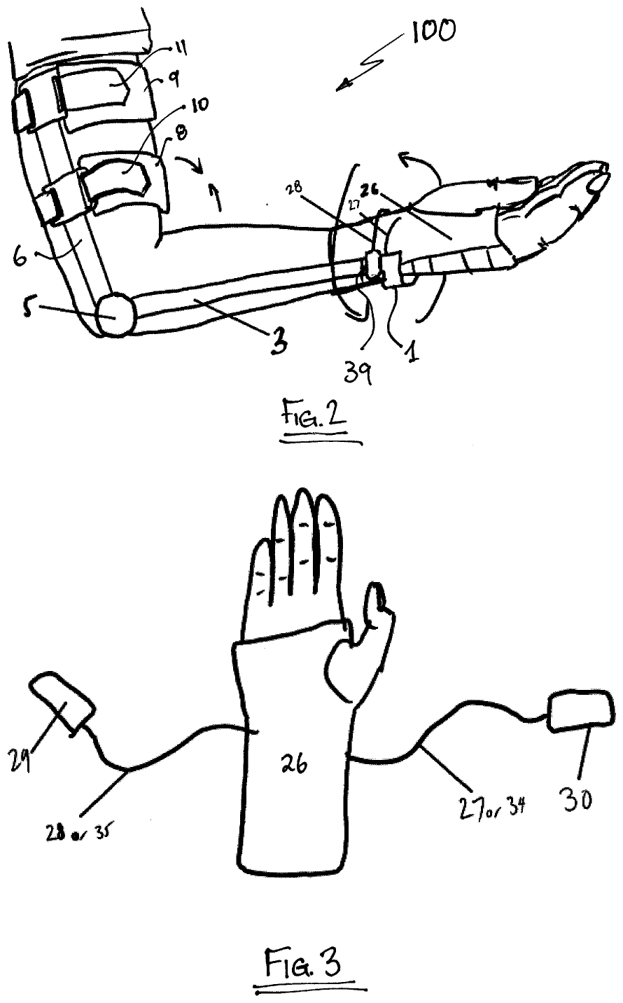

Referring to FIG. 2, a side view of the orthosis 100 as fitted to a wearer, is shown. The wearer is wearing a wrist orthosis 26 with the cords 27, 28 of the wrist bandage attached to the fasteners 28, 29 of the forearm outriggers 2, 3 such that the wearer's wrist is in supination position, with the palm facing upwards. In this position, the orthosis 100 offers the wearer free elbow flexion and extension movements and dynamic forearm supination. The wearer may be able to pronate against the dynamic supination effected by the orthosis.

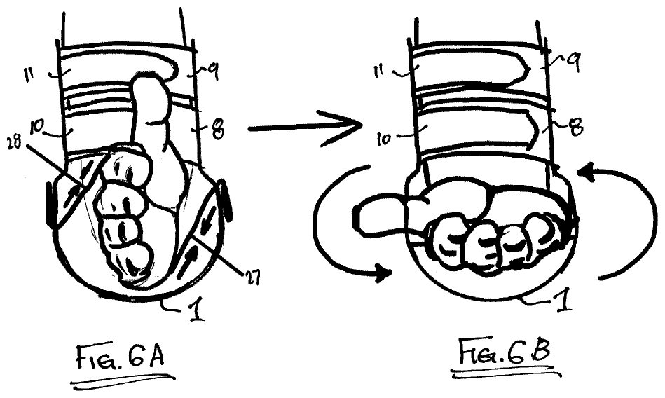

FIG. 5 is a lateral view of a right arm embodied with dynamic elbow flexion and dynamic forearm supination. The dynamic elbow flexion is effected across the hinge articulations 4, 5 by the addition of a spring or elastic member 33 that applies a biasing force to the wearer's elbow in the direction of the arrows, as shown. Dynamic forearm supination is effected as previous by the fastening of the cords 27, 28 to the fasteners 38, 39 located at the ends of the forearm outriggers 2, 3. This is achieved by rotating the wearer's wrist such that the hand is in supination direction and passing the cords 27, 28 around the wrist and attaching to the opposing forearm outrigger 2, 3. The proximal arm padding and strap 9 and 11 is undone to show a mechanism by which the proximal cuff may be attached to the wearer's arm. To pronate rather than supinate, the rotation attachment directions are reversed (not shown). To supinate the left arm, the rotation attachments of the cords 27, 28 would be attached in a mirrored orientation. To extend the elbow, the spring or elastic member 33 is reversed.

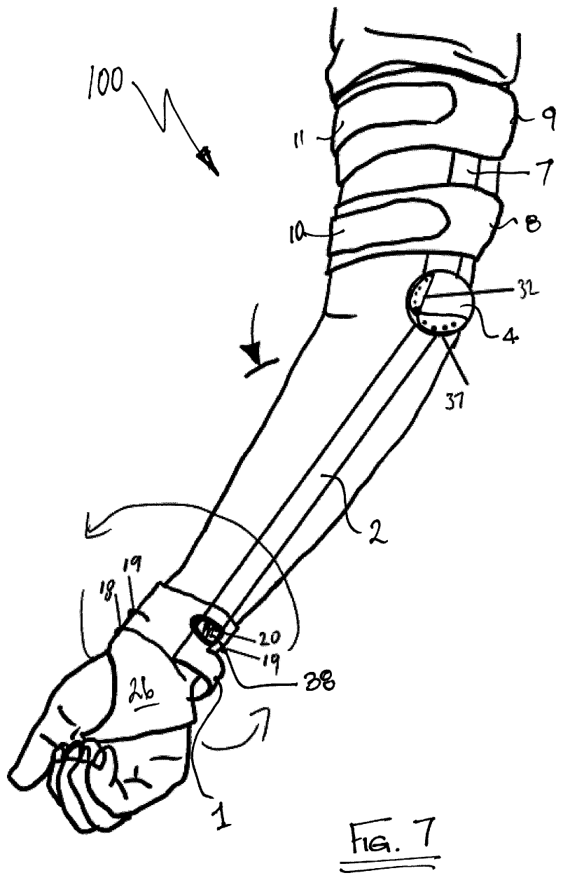

Referring to FIGS. 6A and 6B, a coronal plane view of an arm in mid elbow flexion with elastic cords 27, 28 centring the wrist bandage 26 or wrist cuff 23 between forearm outriggers 2, 3 and the outrigger connection 1. The elastic cords 27, 28 pull the wearer into supination as shown in FIG. 6B. However, they may actively oppose into pronation as is shown in FIG. 6A. The cords 17, 18 are configured to effect dynamic supination as in FIG. 5. If the cords 17, 18 are reversed, the orthosis effects dynamic pronation.

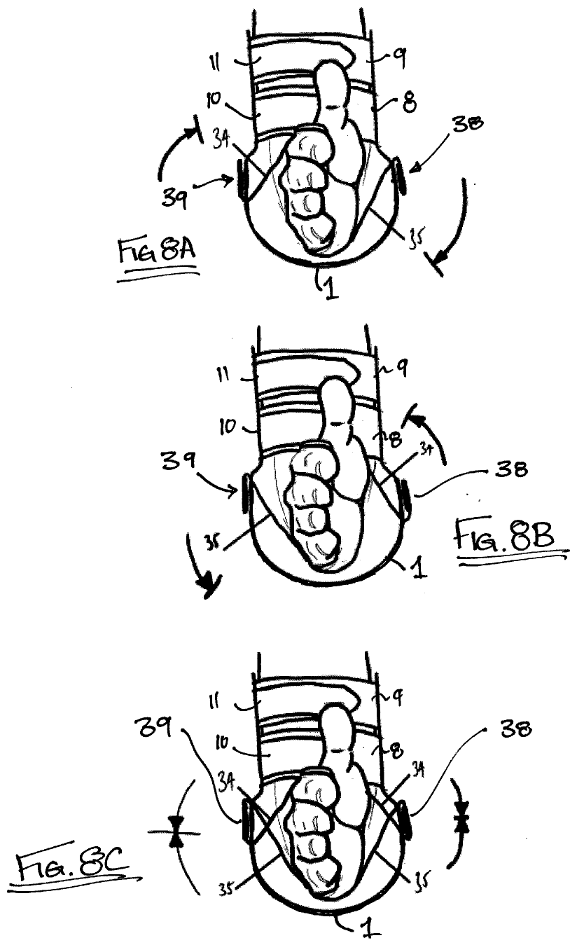

FIG. 7 is a lateral view of an elbow blocking mechanism limiting extension 37 and flexion 32 which may be employed for use with the orthoses 100 of the present invention to facilitate elbow extension limitation and dynamic forearm supination. Such a device is able to integrate with the hinge articulation 4, 5 to vary its function.

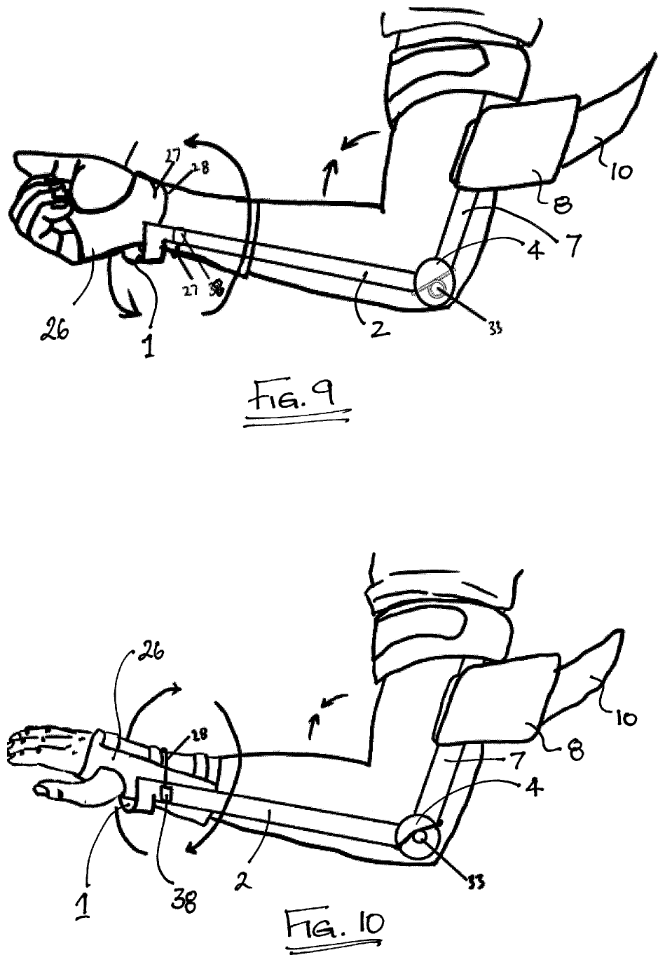

Referring to FIGS. 8A-8C, a coronal plane view of the orthosis in mid elbow flexion with rotation blocking configurations is depicted. In FIG. 8A, pronation may be blocked by the use of inelastic or taut elastic cords 34 and 35 wound in a supinating direction. In FIG. 8B, supination may be blocked by the use of inelastic or taut elastic attachments 34 and 35 wound in a pronating direction. In FIG. 8C, both supination and pronation may be blocked by the use of inelastic or taut elastic attachments 34 ×2 and 35 ×2 wound in both directions of rotation.

It will be appreciated from the above comments, that the orthosis of the present invention can be simply adapted to provide a variety of different therapy options to the wearer. The hinge articulation 4, 5 can be simply adapted to provide elbow flexion or extension as may be required, and limiters can be placed on the hinge articulation 4, 5 to limit the degree of elbow movement possible. Similarly elastic and non-elastic cords can be used to retain the hand in supination or pronation, depending on the desired therapy required. Such an orthosis offers multiple benefits to those requiring corrective therapy to address an injury and/or to recover a movement.

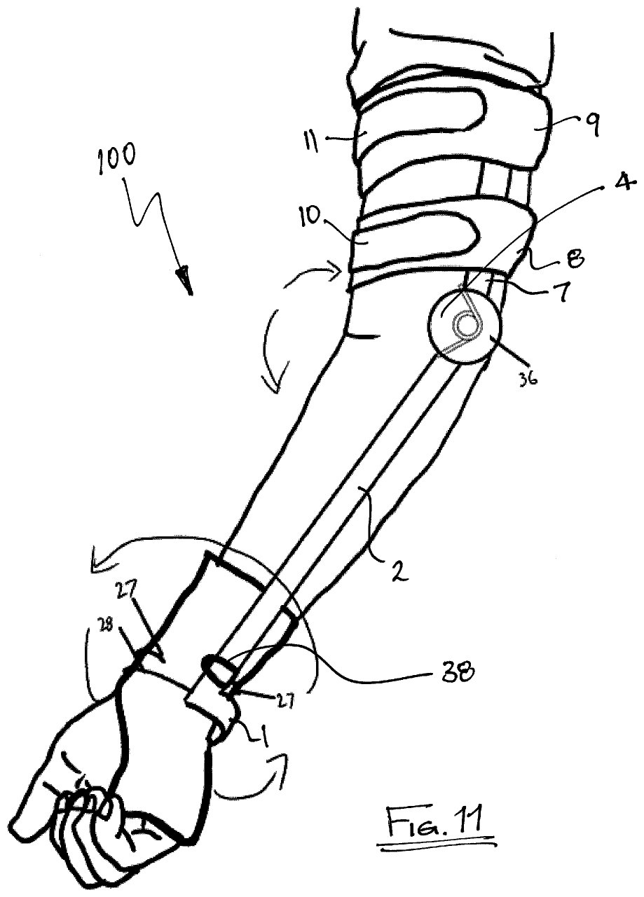

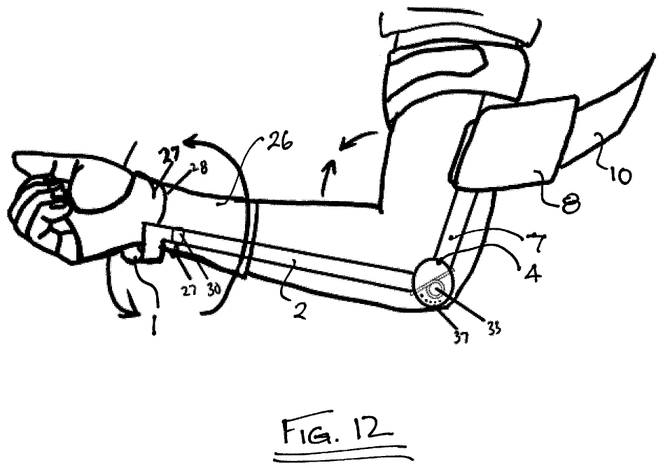

In this regard, FIG. 9 depicts how the placement of a flexion mechanism 33 in the hinge articulation 4, 5 and the use of elastic cords 27, 28 to position the wearer's hand in a supination position enables the orthosis to be simply adapted to provide dynamic elbow flexion and dynamic forearm supination. Similarly, FIG. 10 depicts how the placement of a flexion mechanism 33 in the hinge articulation 4, 5 and the use of elastic cords 27, 28 to position the wearer's hand in a pronation position enables the orthosis to be simply adapted to provide dynamic elbow flexion and dynamic forearm pronation. FIG. 11 depicts how the placement of an extension mechanism 36 in the hinge articulation 4, 5, which provides a bias force to cause the elbow to extend, and the use of elastic cords 27, 28 to position the wearer's hand in a supination position enables the orthosis to be simply adapted to provide dynamic elbow extension and dynamic forearm supination.

The orthosis of the present invention provides elongated members parallel with the forearm that are able to follow the wrist and in doing so, provide a lever for applying rotational movement to the wrist. The elongated members are stabilised distally by way of a connector at the level near the distal radioulnar joint. The hingedly connection between the first and second portions of the orthosis allows the elongated members to follow the wrist regardless of elbow position. If adjusted to and not extend past the hand, it also does not limit hand use. An external rotational force can be achieved via elastic or inelastic attachments linking the outriggers to the wrist or forearm. With the attachments working in the same direction, the wrist is centralised to the elongated members, and the outriggers follow accordingly. The elongated member connector keeps the forearm outriggers from rotating with the wrist. By remaining low profile and proximal to the wrist, the elongated members and the connector do not limit grasp, pinch or hand use. If wrist flexion, extension or deviation is desired, a cuff which allows this may be fitted rather than a wrist bandage.

Further, by following parallel to the forearm, the elongated members produce a torque via the elastic or inelastic cords used to tether the wrist. This provides effective leverage for stretching, assisting or blocking rotation movement. Further, by choosing a flexible or elastic cord or tether, the wearer can move into the opposite direction to the force for functional tasks if desired.

Allowing elbow movement either in active, dynamic or blocking embodiments allows the wearer to function or move the elbow within its desired range/s of motion. If desired, a dynamic element may also be added to the elbow which can concurrently stretch a stiff or assist a weak elbow into either extension and/or flexion. Thus, through simple adaptations, the orthosis of the present invention is able to control or move through both elbow flexion and extension and supination and pronation arcs simultaneously. The orthosis according to the present invention achieves elbow flexibility or control while maintaining the arm as a strong proximal lever for rotation.

Throughout the specification and claims the word “comprise” and its derivatives are intended to have an inclusive rather than exclusive meaning unless the contrary is expressly stated or the context requires otherwise. That is, the word “comprise” and its derivatives will be taken to indicate the inclusion of not only the listed components, steps or features that it directly references, but also other components, steps or features not specifically listed, unless the contrary is expressly stated or the context requires otherwise.

Orientational terms used in the specification and claims such as vertical, horizontal, top, bottom, upper and lower are to be interpreted as relational and are based on the premise that the component, item, article, apparatus, device or instrument will usually be considered in a particular orientation, typically with the orthosis apparatus uppermost.

It will be appreciated by those skilled in the art that many modifications and variations may be made to the methods of the invention described herein without departing from the spirit and scope of the invention.

1. An orthosis comprising:
a first portion adapted to be secured to an upper arm region of a wearer;
a second portion hingedly connected to the first portion and comprising a pair of elongate members spaced apart to extend along opposing sides of a lower arm region of the wearer, each of the elongate members terminate at a distal end adjacent to a wrist of the wearer and being connected by a connecter extending at least partially around the wrist of the wearer;
wherein, the distal ends of the elongated members each have a connector element formed therein for facilitating engagement with the wearer's hand so as to maintain the wearer's hand in either a pronated or a supinated position.

2. An orthosis according to claim 1, wherein the wearer's hand comprises a wrist worn element having one or more attachment members extending from opposing sides thereof for engaging with the connector elements of the elongate members so as to maintain the wearer's wrist in either a pronated or a supinated position when relaxed.

3. An orthosis according to claim 2, wherein the wrist worn element is a soft orthosis.

4. An orthosis according to claim 2, wherein the wrist worn element is a semi rigid orthosis.

5. An orthosis according to claim 1, wherein the wrist worn element is a rigid orthosis.

6. An orthosis according to claim 3, wherein the one or more attachment members are elastic.

7. An orthosis according to claim 6, wherein the elastic attachment members allow movement of the wearer's wrist in an opposing direction when the wearer is active and return to the desired supination or pronation position when the wearer is relaxed.

8. An orthosis according to claim 3, wherein the one or more attachment members are substantially inelastic or are taught elastic.

9. An orthosis according to claim 8, wherein the inelastic attachment members maintain the wearer's wrist in a chosen degree/s of rotation including supinated only, pronated only, restricted supination, restricted pronation or restricted in both supination and pronation.

10. An orthosis according to claim 2, wherein the wrist worn element is a wrist cuff having one or more elastic attachment members extending from opposing sides thereof for engaging with the connector elements of the elongated members so as to maintain the wearer's wrist in either a pronated or a supinated position when relaxed.

11. An orthosis according to claim 10, wherein the elastic attachment members allow movement of the wrist in an opposing direction when the wearer is active and return the wrist to a desired supination or pronation when relaxed.

12. An orthosis according to claim 2, wherein the wrist worn element is a soft wrist cuff having one or more inelastic or taut elastic attachment members extending from opposing sides thereof for engaging with the connector elements of the elongated members so as to maintain the wearer's wrist in a chosen degree/s of rotation.

13. An orthosis according to claim 12, wherein the chosen degree of rotation may include supinated only, pronated only, restricted supination, restricted pronation or restricted in both supination and pronation.

14. An orthosis according to claim 1, wherein the hinged connection between the first and second portions allows free movement of the elongated members in an elbow flexion and extension plane, thereby allowing free movement of wrist cuff or orthosis in this plane.

15. An orthosis according to claim 1, wherein the hinged connection between the first and second portions comprise a flexion spring mechanism for effecting flexion force on the wearer's elbow to maintain the wearer's elbow in flexion and yet allow the wearer to extend their elbow against the flexion force.

16. An orthosis according to claim 1, wherein the hinged connection between the first and second portions comprise an extension spring or elastic mechanism for effecting an extension force on the wearer's elbow to maintain the wearer's elbow in extension and yet allow the wearer to flex against the extension force.

17. An orthosis according to claim 1, wherein the hinged connection between the first and second portions comprise one or two stopping mechanism/s to limit the wearer's elbow flexion and/or extension so as to maintain the wearer's elbow in flexion and/or extension and yet allowing elbow movement within the one or two limits.

18. An orthosis according to claim 1, wherein the hinged connection is lockable in any desired position of elbow flexion or extension, so as to lock the wearer's elbow in a desired position.
